(19) United States
(12) Patent Application Publication
(10) Pub. No.: US 2026/0346043 A1
(43) Pub. Date: Nov. 5, 2026

(54) LIGHT-BASED VAGINAL THERAPY DEVICES AND METHODS FOR USE

(71) Applicant: CERN CORP., Trabuco Canyon, CA (US)

(72) Inventor: Gregg A. Klang, Trabuco Canyon, CA (US)

(21) Appl. No.: 19/642,467

(22) Filed: Apr. 8, 2026

Related U.S. Application Data
(63) Continuation of application No. PCT/US24/50601, filed on Oct. 9, 2024.
(60) Provisional application No. 63/543,265, filed on Oct. 9, 2023.

(51) Int. Cl. A61N 5/06; A61K 9/00; A61K 41/00; A61K 47/38

(52) U.S. Cl. CPC A61N 5/0603; A61K 9/0034; A61K 41/0057; A61K 47/38; A61N 5/062; A61N 5/0624; A61N 2005/0611; A61N 2005/0652; A61N 2005/0663

(57) ABSTRACT

Light-based therapy devices, photosensitizers, and kits are provided for treating vaginal bacterial and fungal infections. In one example, the device includes a body sized for introduction into a vagina; and one or more light sources configured to emit light outwardly from the body. A source of photosensitizer including curcumin is provided for use with the device, e.g., to enhance treatment, reduce power consumption, reduce treatment time, and/or reduce overall dosage of light delivered. In one example, the photosensitizer includes a carboxy methyl cellulose (CMC) carrier and curcumin mixed or in suspension with the CMC carrier, the concentration of curcumin being not more than about two millimolar (2.0 mM) or between about half and two millimolar (0.5-2.0 mM). In one example, the light source(s) may be configured to deliver light at wavelengths between about 425-475 nanometers and/or generate power under fifty milliwatts per square centimeter (50 mW.cm2).

### RELATED APPLICATION DATA

The present application is a continuation of co-pending International Application No. PCT/US 2024/050601, filed Oct. 9, 2024, which claims benefit of U.S. provisional application Ser. No. 63/543,265, filed Oct. 9, 2023, and is related to U.S. Pat. Nos. 10,004,918 and 11,207,544, and U.S. application Ser. No. 17/714,914, filed Apr. 6, 2022, the entire disclosures of which are expressly incorporated by reference herein.

### TECHNICAL FIELD

The present application generally relates to medical devices and, more particularly, to light-based vaginal light therapy devices for treatment of bacterial and fungal infections and systems and methods for using such devices.

### BACKGROUND

Vaginitis is characterized by the inflammation of the vagina that results in discharge, itching and pain. The cause is usually a change in the normal balance of vaginal bacteria or an infection. Vaginitis can also result from reduced estrogen levels after menopause. In a given year as many as 75% of the woman female population experiences bacterial or fungal infection within their vagina. The symptoms range from mucus-like discharge, itching, aching, pain during intercourse to odor. The vaginal infections often have multiple causes that present challenging cases for treatment. It is critical to have a balance between naturally occurring yeast and bacteria. It is when the system is out of balance or other types of bacteria are present within the environment does one end up with vaginitis. Indeed, when one cause is treated, the other pathogens become resistant or get mutated when treated with antibiotic and become resistant to antibiotic therapies. Sometimes the reduction in good bacteria allows for a propagation of yeast, typically Candida albicans resulting in yeast infection. Further, either a change in pH balance or introduction of foreign bacteria in the vagina leads to infectious vaginitis. Physical factors that contribute to the development of an infection include the following: constantly wet vulva due to tight clothing, chemicals coming in contact with the vagina via scented tampons, antibiotics, birth control pills, or a diet favoring refined sugar and yeast.

Bacterial vaginosis also known as vaginal bacteriosis or Gardnerella Vaginitis is a disease of the vagina caused by excessive bacteria growth. Common symptoms include increased vaginal discharge that often smells fishlike. The discharge is usually white or gray in color. Burning with urination may also occur. Itching is uncommon. Occasionally there may be no symptoms. Having bacterial vaginosis increases the risk of infection by a number of other sexually transmitted infections including HIV/AIDS. It also increases the risk of early delivery among pregnant women. Bacterial vaginosis is caused by an imbalance of the naturally occurring bacteria in the vagina. Diagnosis is suspected based on the symptom and may be verified by testing the vaginal discharge and finding a higher than normal vaginal pH and large numbers of bacteria. Bacterial vaginosis is often confused with a vaginal yeast infection. Usually treatment is through the use of antibiotics. Bacterial vaginosis is the most common vaginal infection in women of reproductive age. The percentage of women affected at any given time varies between can be as high as 35%. Antibiotics, administered either orally or vaginally are effective in treatment. About 10% to 15% of people, however, do not improve with the first course of antibiotics and recurrence rates of up to 80% have been documented. Recurrence rates are increased with sexual activity with the same pre-post treatment partner and inconsistent condom use although estrogen-containing contraceptives decrease recurrence. There is evidence of an association between Bacterial vaginosis and increased rates of sexually transmitted infections such as HIV/AIDS. Bacterial vaginosis is associated with up to a six-fold increase of HIV shedding. There is also a correlation between the absence of vaginal lactobacilli and infection of Neisseria gonorrhoeae and Chlamydia trachomatis.Bacterial vaginosis is a risk factor for viral shedding and herpes virus type-2 infection. Bacterial vaginosis may increase the risk infection or reactivation of HPV.

Candidiasis, more commonly referred to as a Yeast Infection, is most commonly caused by an overgrowth of a fungus called Candida albicans in the vagina. Candida is yeast, a type of fungus. Yeast is always present in the vagina in small numbers, and symptoms only appear with overgrowth. Candida can multiply when an imbalance occurs, such as when the normal acidity of the vagina changes or when hormonal balance changes. Frequently occurring yeast infections may be a sign of more serious overarching health problem such as diabetes or a compromised immune system. Recurrent infections may also be due to use of antibiotic medications. Recurrent vulvovaginal candidiasis affects at least 75 million women annually in the U.S. About 5-8% of women experience four or more episodes per year, diagnosed as recurrent vulvovaginal candidiasis. About 75% of all premenopausal women develop thrush at some point in their lives. With the introduction of over-the-counter medications for home treatment of yeast infections, many women elect to self-diagnose and self-medicate, indicating that the true incidence of yeast infections annually may be significantly under-reported.

In comparison to antibacterial therapy, antifungal treatment is limited to a very small number of drug substances. Treatment for fungal infection can be topical or systemic. Topical antifungals are generally considered as first-line therapy for uncomplicated, superficial, relatively localized fungal infections due to their high efficacy and low potential for systemic adverse effects. Systemic antifungal agents are absorbed and delivered to the body through the blood stream. The oral route is usually the safest, the most economical, and the easiest route for systemic antifungal drugs.

Topical antifungal creams and suppositories have fewer side effects than oral antifungal medications because they aren't absorbed as readily, systemically by the body, and only exert a localized effect on the genital region. Antifungal pills affect the entire body, and side effects can include nausea, headaches, and abdominal pain. However, topical medications can be messy and uncomfortable, while pills are comparatively simple. Treatment using antifungal medication is ineffective in up to 20% of cases. Treatment for thrush is considered to have failed if the symptoms do not clear within 7-14 days. In addition, the incidence of resistance to antifungal agents may be increasing, with drug-resistant fungal strains becoming increasingly common causes of infection in high-risk patient groups such as HIV/AIDS patients. Accordingly, alternative antifungal strategies are being actively sought.

Severe forms of infection are hard to treat, and frequently require more aggressive and long-term therapy, as is the case with chronic, recurrent cases. Additionally, incomplete treatments often result in drug resistant infections therefore full course of therapy should be adhered to.

Although, light therapy treatment of various bacterial, fungal or viral infection is generally known, a treatment of such infections is generally achieved through chemical or drug therapies. A use of such therapies affects an internal functioning of the vagina and uterus as the chemicals used in the form of paste or gel or liquid result in unwanted chemical reactions that are harsh or result in various complications.

Oral antifungal medications carry the risk of significant side effects, and many patients are allergic to or intolerant of these drugs. Topical solutions can be messy and inconvenient. There are no existing products for the treatment of yeast infections without also requiring medication. Hence there is a need for a product that allows for the treatment of yeast and bacterial infections quickly and simply without systemic effects. With the continued and accelerating emergence of antibiotic-resistant microorganisms, there is burgeoning interest and investment in light therapy. A device that leverages this rising technology could potentially gain rapid acceptance in specific use cases as well as broader support among the general population simply wishing to avoid exposure to additional medications.

In the view of the foregoing, devices and methods for treating intravaginal infections would be useful.

### SUMMARY

The present application generally relates to medical devices and, more particularly, to light-based vaginal light therapy devices for treatment of bacterial and fungal infections and/or for preventing of such infections, e.g., before/during pregnancy, and to systems and methods for using such devices.

In accordance with one example, a device, system, or kit is provided for vaginal light therapy of a user that includes a housing comprising a body sized for introduction into a vagina; one or more light sources configured to emit light outwardly from the body; and a source of photosensitizer comprising curcumin.

In accordance with another example, a device, system, or kit is provided for vaginal light therapy of a user that includes a housing comprising a body sized for introduction into a vagina; one or more light sources configured to emit light outwardly from the body; and a source of photosensitizer comprising a carboxy methyl cellulose (CMC) carrier and curcumin mixed or in suspension with the CMC carrier, the concentration of curcumin being not more than about two millimolar (2.0 mM).

In accordance with still another example, a photosensitizer is provided for use with a light therapy device to deliver vaginal light therapy to a user to treat one or more pathogens that includes carboxy methyl cellulose (CMC) carrier; and curcumin mixed or in suspension with the CMC carrier, the concentration of curcumin being not more than about two millimolar (2.0 mM), e.g., having a concentration of curcumin between about half and two millimolar (0.5-2.0 mM).

In accordance with yet another example, a method is provided for vaginal light therapy of a user that includes providing a therapy device comprising body comprising one or more light sources; providing a photosensitizer comprising curcumin on or in conjunction with the body; inserting the therapy device into a vagina; and activating the one or more light sources to emit light outwardly from the body.

In one example, the devices herein may include a housing comprising a body sized for introduction into a vagina and including a central region and tapering towards a distal end, and, optionally, an appendage integrally formed with the body and extending proximally from the body, the appendage having sufficient length to extend from the vagina when the body is fully inserted into the vagina and formed from malleable material such that the appendage may be manipulated to a desired shape and retain the desired shape; one or more light sources configured to emit light outwardly from the body; and a battery configured to deliver electrical power to activate the one or more light sources.

These and other aspects of the examples herein will be better appreciated and understood when considered in conjunction with the following description and the accompanying drawings. It should be understood, however, that the following descriptions, while indicating examples and numerous specific details thereof, are given by way of illustration and not of limitation. Many changes and modifications may be made within the scope of the examples herein without departing from the spirit thereof, and the examples herein include all such modifications.

### BRIEF DESCRIPTION OF THE DRAWINGS

The other objects, features and advantages will occur to those skilled in the art from the following description of the examples and the accompanying drawings in which:

FIG. 1A illustrates a perspective view of a light-based vaginal light therapy device.

FIG. 1B illustrates a perspective view of a light-based vaginal light therapy device with a USB cord.

FIGS. 1C, 1D, and 1E are top, side, and front views, respectively, of the device of FIG. 1B.

FIG. 1F illustrates a sectional view of the light-based vaginal light therapy device of FIG. 1B.

FIG. 2 illustrates placement of a vaginal light therapy device inside a vaginal canal of a female.

FIG. 3 is a cross-sectional view of an example of a cradle for storing, charging, and/or otherwise receiving a light therapy device, such as the device shown in FIGS. 1A-1F showing exemplary components of a light therapy device and cradle.

FIGS. 4A and 4B are perspective views of an example of a cradle with a light therapy device stored therein, showing a lid of the cradle open and closed, respectively.

FIGS. 5A and 5B are perspective view of another example of a cradle showing a lid of the cradle open and closed, respectively.

FIGS. 6A and 6B are perspective view of another example of a cradle showing a lid of the cradle open and closed, respectively.

FIG. 7 is a perspective view of an insertion tool that may be provided onto which a treatment device, such as that shown in FIGS. 1A-1F, may be loaded to facilitate insertion into a user's vagina.

FIG. 8 is a perspective view of another example of a light-based vaginal light therapy device.

FIGS. 9A-9D show yet another example of a light-based vaginal light therapy device.

FIG. 10 is a schematic showing exemplary components that may be included in the device of FIGS. 9A-9D.

FIG. 11 is a perspective view of still another example of a light-based vaginal light therapy device.

FIGS. 12A and 12B show another example of a system for light-based vaginal therapy that includes a therapy device sized for introduction into a vagina and an external controller that may be coupled to the therapy device, e.g., to deliver light into the therapy device via a fiberoptic cable for delivering light therapy within the vagina.

### DETAILED DESCRIPTION

In the following detailed description, reference is made to the accompanying drawings that form a part hereof, and in which the specific examples that may be practiced is shown by way of illustration. The examples are described in sufficient detail to enable those skilled in the art to practice the examples and it is to be understood that the logical, mechanical and other changes may be made without departing from the scope of the examples. The following detailed description is therefore not to be taken in a limiting sense.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

The examples herein provide a light-based vaginal light therapy device that may be used to treat a variety of conditions, e.g., bacterial and fungal infections, chlamydia, and the like. Alternatively, the devices and systems herein may also be useful for applying other forms of energy to the vaginal wall, e.g., to tighten adjacent tissues. In one example, the device may include a body or housing, a single or a plurality of light sources, a switch or other actuator, a flexible tether, a microchip or other controller, and a battery or other power source. Alternatively, the device may include a wireless communications interface, e.g., including one or more antennas and/or circuits, for receiving commands from a remote electronic device, e.g., the user's mobile phone, tablet, or other device, via Bluetooth or other communications protocols.

Optionally, the body of the devices herein may be formed from an appropriate medical grade, durable material which allows the therapeutic light to be emitted. Optionally, one end of the device may include a cervix support to place the device against the cervix. In one example, the light source(s) may include a single or multiple LEDs provided over the LED body, e.g., mounted to the outer surface, within recesses in the outer surface, and the like, e.g., to provide a substantially smooth and/or atraumatic outer surface for the device. Alternatively, other internal light sources may be provided that may transmit light from the body, e.g., via one or more fiber optics, lenses, through a transparent (to the transmitted light) wall of the body, and the like (not shown). For example, the body may be formed entirely of transparent material or desired regions may be transparent such that one or more LEDs within the body may transmit light through the transparent material to treat adjacent tissue. Alternatively, an external light source, e.g., within a controller, may be provided that delivers light to the device via one or more fiberoptic cables, as described further elsewhere herein.

Each LED may emit a light with a wavelength in a therapeutic zone of light in a range of blue and/or red light or violet light (e.g., germicidal non-UV) wavelength. In an example, the switch is a pressure activated switch. The controller is housed within the body and connects a battery to the light source(s) and is further connected to the switch. The controller may control the duration of light therapy and/or pulse the light. Alternatively, the battery charge may be set to limit the duration and/or other functionality of the device. For example, pulsing the light may stress the targeted bacteria or yeast and/otherwise make the device more effective. The switch and the light source(s) draw power from the battery through the microchip. The switch controls an activation as well as deactivation of the light source(s).

The tether, e.g., a flexible cable, rope, cord, loop, and the like, may be connected to one end of the body and may have sufficient length to facilitate retrieving and/or progressing the device from a vagina. For example, the tether may be a flexible cord including first and second ends coupled to the body, e.g., to define an enclosed loop having sufficient length to extend out of a vagina when the body is inserted entirely into the vagina, e.g., against a cervix. Alternatively, a cannula or other insertion tool (not shown) may be provided for inserting and/or retrieving the device.

According to examples herein, the light source(s) may emit a non-UV germicidal light with a wavelength ranging between a blue light wavelength and/or a red light wavelength or a Violet light wavelength. For example, LEDs may be used that emit light in the range of 405 nm-470 nm. Alternatively, the LEDs emit light in the range of 620 nm-750 nm, or the LEDs may emit light in the range of 380 nm-450 nm. The emitted light may kill or limit propagation of various strains of bacteria and fungus or other pathogen contributing to infection or virus.

According to one example, the controller controls the duration of light pulses in a rapid on and/or off manner. For example, once activated, the controller may maintain the light source(s) active for a predetermined time period, e.g., an hour or more, and then automatically deactivate the light source(s). Optionally, when the device is activated, the controller may pulse the light source(s), e.g., rapidly turning the light source(s) off and on multiple times per minute or per second. In addition or alternatively, the light source(s) may be pulsed between different wavelengths.

According to one example, the device is non-reusable and serves a treatment of bacterial and fungal infection for single use. Alternatively, the device is reusable and may be used for treatment of bacterial and fungal infection multiple times and may include or be coupled to a cable and for recharging the device. The reusable device has a washable or rinse-able body.

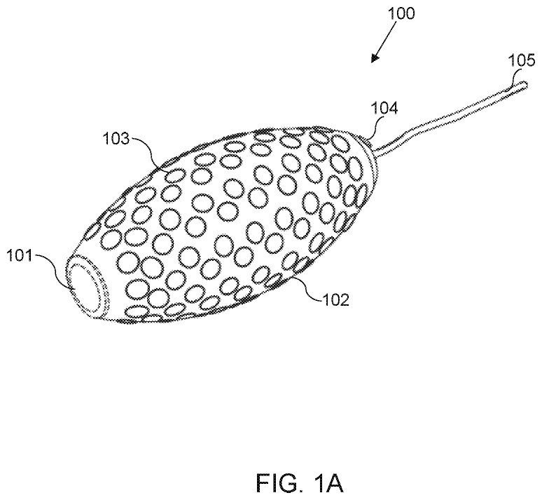

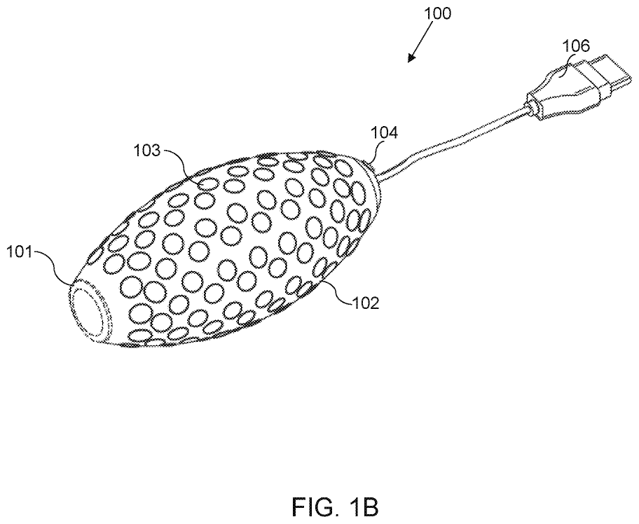

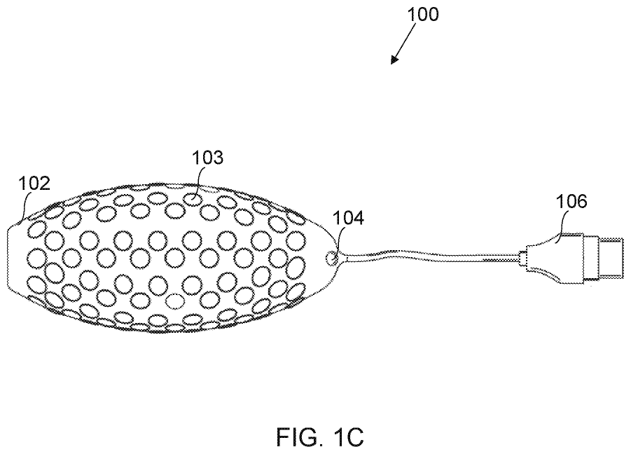

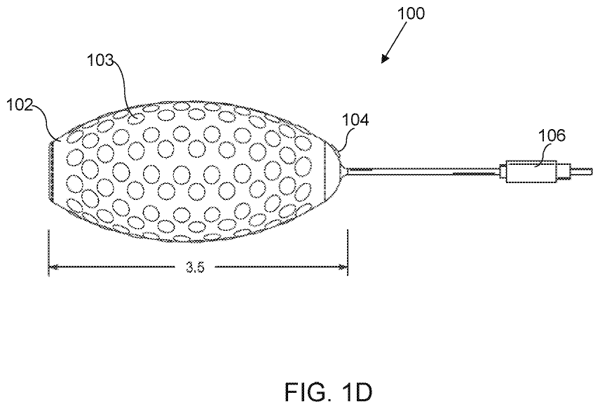

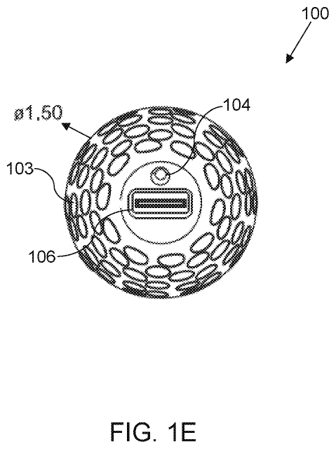

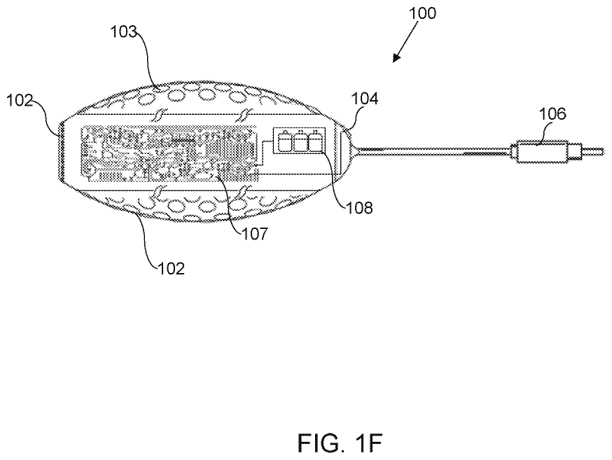

Turning to the drawings, FIG. 1A illustrates a perspective view of an example of a LED-based vaginal light therapy device 100 including a body 102 carrying a plurality of light sources 103. FIG. 1B illustrates a perspective view of the LED-based vaginal light therapy device 100 with a USB cord 106. FIGS. 1C-1F illustrates a top view, a side view, a front view and a sectional view respectively of the LED-based vaginal light therapy device 100, according to one example. With respect to FIGS. 1A-1F, the body 102 of the light therapy device 100 is primarily a plastic framework which allows positioning of single or multiple LEDs 103 on an external surface. As shown in FIG. 1F, a battery 108, controller 107, and/or additional electronic controllers and circuits are positioned internally with respect to the LEDs 103.

A switch 104 may be located on the outer surface of the body 102, which may be actuated by the user to activate the device 100, e.g., before insertion into the vagina. Alternatively, the tether 105 may be coupled to a switch within the body such that the tether 105 may be pulled or otherwise manipulated to activate/deactivate the light sources 104. In a further alternative, a pressure-activated switch (not shown) may be provided within the body 102 that is responsive to compressive pressures on the body 102, e.g., such that the device 100 may automatically activated once inserted into the vagina and the pressure from the surrounding muscles compress the body 102.

The device 100, once assembled, is encased into an appropriate medical grade plastic housing which is completely sealed until not serviceable. A suitable tether 105, as shown in FIG. 1A, or a mini-USB cable 106, as shown in FIG. 1B, may be attached at a first or proximal end of the body 102 to assist in insertion and/or retrieval of the device 100 inwards or outwards of the vaginal canal. Optionally, a second or distal end of the device 100 may include a cervix support 101, e.g., a concave or otherwise shaped recess to place the device smoothly against the cervix.

In one example, the length and diameter of the body 102 may be sized for insertion fully into a vagina, e.g., having a generally elliptical or oblong shape, i.e., with the length greater than the diameter. For example, the body 102 may have a length not more than about 3.5 inches and a maximum diameter, e.g., at a central region of the body 102, of not more than about 1.5 inches. Optionally, the body 102 may be available in multiple sizes, e.g., lengths and/or diameters, which may be provided to individual patients or users based on their individual anatomy. Thus, the device may have any appropriate size so as to address the size of the cavity in which it is inserted, e.g., to seat the device against the cervix and/or otherwise minimize migration during use. The surface of the body 102 is either rigid or squeezable depending on the basis of user preference and area of usage. Alternatively, the material of the body 102 may be flexible and/or malleable to allow the body 102 to conform to a vaginal cavity into which the device 100 is inserted. In one example, the body 102 has an ellipsoid shape, e.g., including a rounded proximal end, a distal end, a relatively large diameter central region substantially midway between the proximal end and the distal end, a proximal tapered region tapering from the central region to the proximal end, and a distal tapered region tapering from the central region to the distal end.

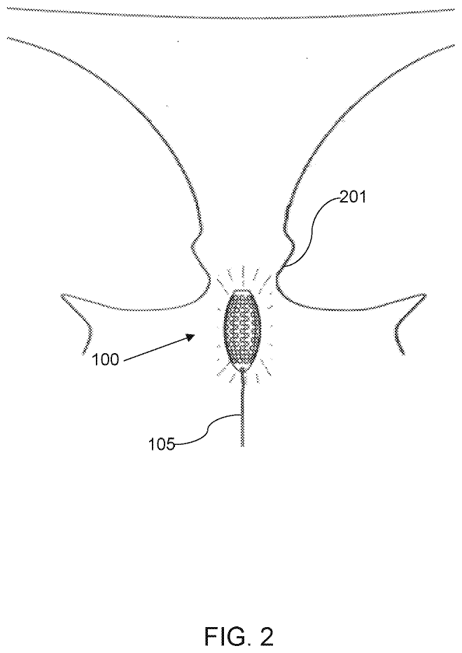

FIG. 2 illustrates a placement of the vaginal light therapy device inside the vaginal canal of a female. Although device 100 is shown as an example, the methods described herein are equally applicable to all of the devices described herein. With respect to FIG. 2, the device 100 is inserted fully into the vaginal canal 201, e.g., to place the distal end against the cervix and with the proximal end receive within the vaginal canal 201, such that the tether 105 or USB cable 106 extends from the vaginal canal 201.

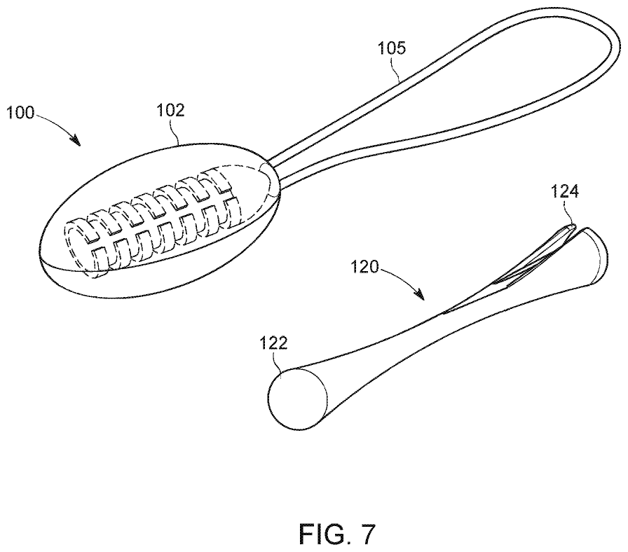

Optionally, as shown in FIG. 7, an insertion tool 120 may be provided to facilitate introduction of the device 100 into the vaginal canal. Generally, the insertion tool 120 includes a proximal end 122, e.g., including a handle (not shown) and/or shaped to facilitate manipulation of the insertion tool 120, and a distal end 124, e.g., including one or more connectors and/or features for releasable engaging the proximal end of the body 102. Optionally, the insertion tool 120 may include a recess, e.g., an elongated groove extending proximally from the distal end 124 sized for receiving the tether 105 of the device 100.

For example, the distal end 124 may have a concave shaped recess corresponding to the shape of the body 102 such that the body may be seated partially in the recess, whereupon the tether 105 may be inserted into the groove. Optionally, the groove may provide sufficient interference fit with the tether 105 to prevent the tether from falling out and/or holding the body in place against the distal end 124. Alternatively, the insertion tool 120 may include a post, hub, or other element (not shown) over which the tether 105 may be looped or wrapped one or more times to secure the tether 105 to the insertion tool 120, e.g., with sufficient tension to hold the device 100 on the distal end 124.

In addition or alternatively, the insertion tool 120 may include one or more fingers, detents, or other features (not shown), which may be received within corresponding features in the body 102 to secure the device 100 to the distal end 124. In this example, the features may be releasable, e.g., using a button or other actuator (not shown) on the proximal end 122 of the insertion tool 120 to allow the device 100 to be released once positioned within the cavity.

The insertion tool 120 may be formed from substantially rigid or malleable biocompatible material, e.g., metal, plastic, or composite material, having sufficient length to allow the distal end 124 to be inserted into the vaginal canal while holding the proximal end 122 outside the user's body, e.g., between about five and six inches (12.5-15 cm). The insertion tool 120 may be substantially straight or may have a desired curved shape between the proximal and distal ends 122, 124 to facilitate use.

Returning to FIG. 2, before or after introduction, the device 100 may be activated, e.g., by activating an accelerometer-controlled switch, by actuating a mechanical switch 104 on the body 102 before inserting the device 100 into the vagina, and the like, as described elsewhere herein, or the device 100 may be pressure-activated, e.g., after it reaches a predetermined position in the vaginal canal 201, whereupon the light source(s) start emitting the light. Alternatively, if the device includes a wireless communications interface (not shown), the device may be controlled wirelessly, e.g., using an appropriate interface, e.g., a phone-based application that may be downloaded on an electronic device (not shown) of the user. The device 100 is left in the vagina for a specific period of time varying from a few minutes to hours depending upon extent of infection and kind of infection (e.g., bacterial, fungal, or other infections). During the treatment period, the user may resume normal activities given the relatively small size and comfortable shape of the device 100.

The light therapy devices 100 disclosed herein may provide a harmless and/or efficient treatment of the intravaginal infection. Since the device 100 does not react with any vaginal fluid, the device 100 may be used in any patient's condition. Also the device 100 may have a relatively low cost and easy usage procedure, so it is usable even personally after a physician's PATENT approval. Optionally, one or more features of the device may be provided to address concerns such as overuse and/or overexposure. For example, the capacity of the battery may be selected to limit the maximum time period during which the device may be activated and/or to require a minimum recharge time or such parameters may be automatically controlled by the controller within the device.

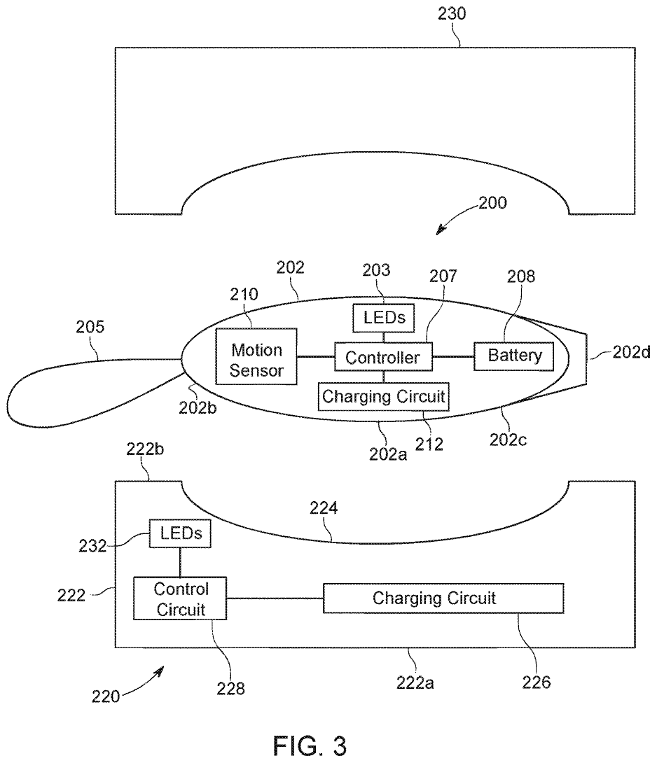

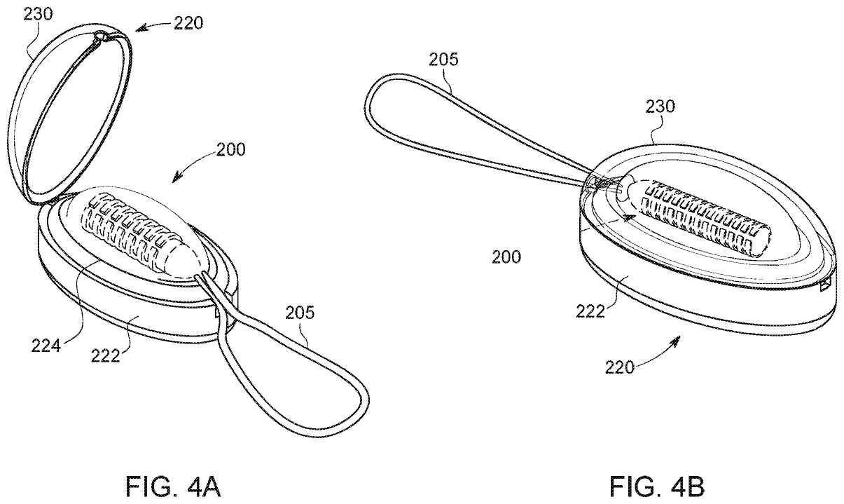

Turning to FIGS. 3 and 4, another example of a light therapy device 200 is shown that is constructed generally similar to the previous examples, e.g., including an oblong-shaped body 202 containing internal components of the device 200 within a sealed environment and a flexible tether 205 extending from one end of the body 202. Similar to other examples herein, the body 202 may include a central region 202a defining a maximum diameter that tapers to proximal and distal ends 202b, 202c. As shown, the proximal end 202b is rounded while the distal end 202c includes a cervix support surface 202d, e.g., having a flat shape as shown, or a concave, convex, or other shape (not shown) that may facilitate placement of the body 202 within a vagina against the cervix (not shown).

Generally, the device 200 includes one or more light sources, e.g., one or more LEDs 203, a controller 207, and a battery 208, similar to the previous examples. In addition, the device 200 includes an accelerometer or other motion sensor 210 within the body 202 that is coupled to the controller 207 instead of an external switch. For example, the controller 207 may monitor signals from the motion sensor 210 to identify predetermined commands, e.g., to activate or deactivate the LEDs 203, and/or direct the device 200 through one or more operational modes. Exemplary motions may include moving the body back-and-forth in a linear motion, spinning the body, and the like. In one example, a first distinct motion or set of motions may be identified by the controller 207 to toggle the device 200, i.e., alternately activating and deactivating the LEDs 203. A second distinct motion or set of motions may be identified to direct the controller 207 to modify the activation between a menu of options, e.g., between continuous and one or more pulsed activation profiles, changing light frequency transmitted by the one or more light sources, and the like. Thus, each time the second motion is repeated, the controller 207 may modify operation of the LEDs 203 between the sequence of options. Alternatively, a distinct motion may be assigned to each desired command.

In addition or alternatively, the device 200 (or any of the other devices herein) may include an inductive charging circuit 212 within the body 202, e.g., coupled to the battery 208. In one example, the charging circuit 212 may include one or more magnets, coils, capacitors, and/or other components (not shown) that may be activated by an external magnetic field to generate electrical current to charge the battery 208, as described further elsewhere herein.

As shown in FIGS. 3 and 4, a cradle or case 220 may be provided for storing a light therapy device, such as device 200, when not in use, e.g., as part of a system or kit that may be provided to users. Generally, the cradle 220 includes a housing or base 222 and, optionally, a lid, cover, or other enclosure 230. The base 222 generally includes a planar lower surface 222a for placing the cradle 220 on a table or other surface (not shown), and an upper surface 222b including a cavity 224 sized to receive the device 200. For example, the cavity 224 may define a portion of the oblong shape of the body 202 of the device 200 such that the device 200 may be received at least partially in the cradle 220, e.g., in a predetermined orientation. In one example, the cavity 224 may have an elongated oblong shape, e.g., sized to receive the body 202 sideways such that the proximal and distal ends 202b, 202c of the body 202 are positioned at opposite ends of the cavity 224, as shown in FIGS. 3 and 4. Alternatively, the cavity 224 may have a tapered side wall terminating at a flat, concave, or convex lower surface (not shown), e.g., corresponding to the shape of the cervix support surface 202d of the body 202, such that the device 200 can only be received in the cradle 220 with the cervix support surface 202d inserted first into the cavity 224. Alternatively, the cavity 224 may have other shapes, e.g., a semi- or partial-spherical or other shape (not shown) larger than the maximum dimension of the body 202 such that the device 200 may be placed in the cradle 220 in any orientation.

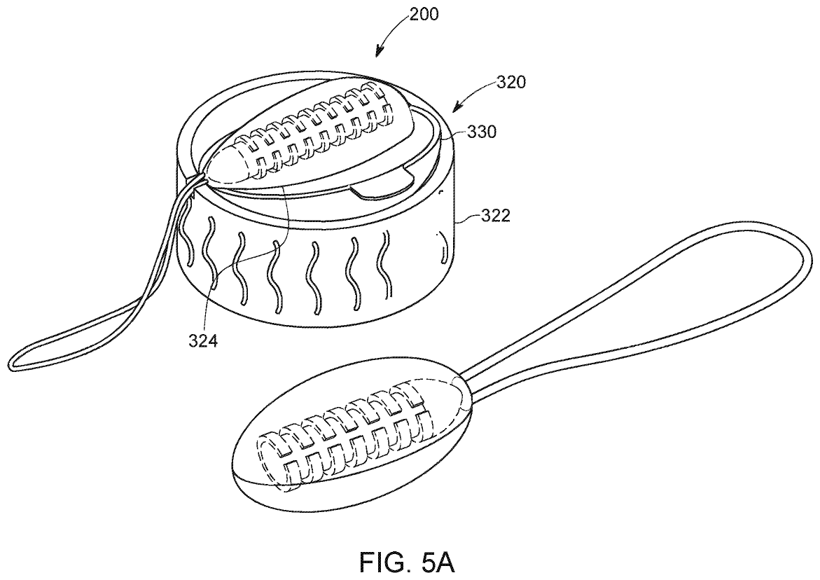

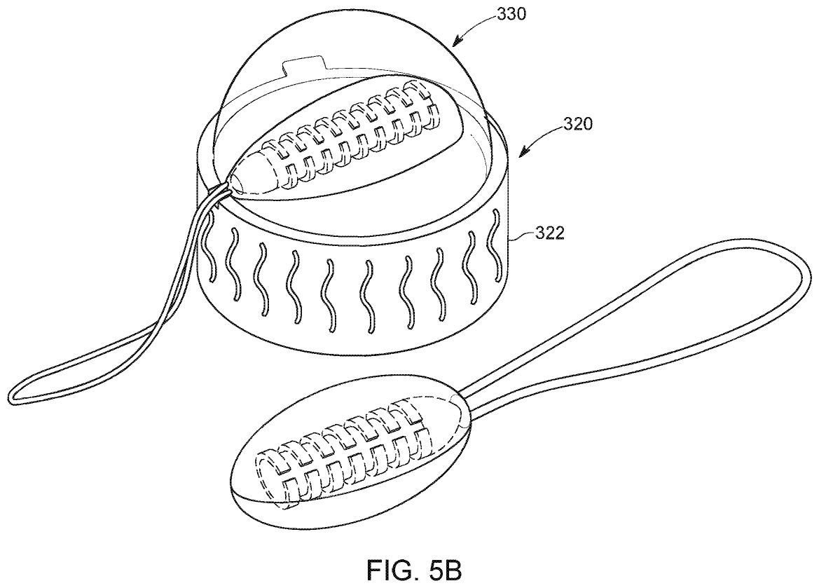

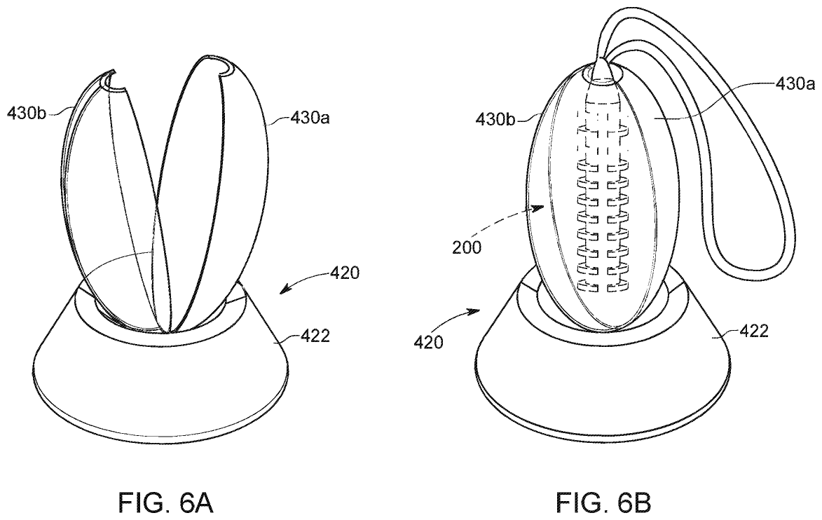

Additional examples of cradles are shown in FIGS. 5 and 6, which may be used with any of the devices described herein. For example, FIGS. 5A and 5B show a cradle 320 including a base 322 having a cavity 324 for receiving a light treatment device 200, and a lid 330 that rotatably slides into the base 322 to allow the deice 200 to be placed into and/or removed from the cavity 324. FIGS. 6A and 6B show yet another example of a cradle 420 including a base 422 and a pair of clamshells or other housing members 430a, 430b that may open and close to receive a light treatment device 200 within a cavity defined by the clamshells 430a, 430b.

Returning to FIGS. 3 and 4, if the cradle 220 includes a lid 230, the lid 230 may include a cavity 232 corresponding to the cavity 224 within the base 222, e.g., to provide an enclosed chamber when the lid 230 is closed over the housing 222. The lid 230 may be coupled to the base 222 by one or more hinges (not shown), e.g., such that the lid 230 may be pivoted between open and closed positions. Alternatively, the lid 230 may be separate from the base 222 and one or more cooperating connectors, e.g., tabs, detents, grooves, and the like (not shown), may be provided that allow the lid 230 to be removably secured over the base 222 to enclose the chamber.

Optionally, the cradle 200 may include one or more features for interacting with the device 200, e.g., as shown in FIG. 3. For example, the cradle 220 may include an inductive charging circuit 226 mounted adjacent the cavity 224 for delivering energy to the charging circuit 212 of a device 200 placed in the cavity 224. For example, the charging circuit 226 may be configured to generate a magnetic field that activates the charging circuit 212 within the device 200, e.g., including one or more magnets, coils, or other components (not shown), to charge a battery of the device.

In one example, the cradle charging circuit 226 may be activated automatically when the device 200 is placed in the cradle 220 or may be selectively activated by the user, e.g., by actuating a switch, button, or other actuator (not shown). For example, the cradle 220 may include a control circuit 228 that periodically activates the cradle charging circuit and identifies when the resulting magnetic field indicates that a device 200 is present in the cavity 224. Once a device 200 is identified, the control circuit 228 may activate the cradle charging circuit 226 for a predetermined time to charge the battery 208 of the device 200.

Alternatively, the controller 207 in the device 200 may include a circuit component (not shown) that modifies the magnetic field or otherwise communicates wirelessly to the cradle control circuit 228 when the controller 207 confirms that the battery 208 has been fully charged. When the cradle control circuit 228 detects the modified magnetic field or other communication from the device controller 207, the control circuit 228 may deactivate the cradle charging circuit 226.

Optionally, the cradle 220 may include one or more features to assist and/or facilitate cleaning a device 200 between uses, e.g., in addition to or instead of the inductive charging circuit 226. For example, as shown in FIG. 3, one or more light sources 232 may be provided on or in the cradle 220 for cleaning the device 200, e.g., applying germicidal and/or bactericidal light at one or more frequency ranges, such as ultraviolet light, or non-ultraviolet germicidal light, and/or otherwise neutralizing latent pathogens on the outer surface of the body 202. In one example where the cradle 220 includes a lid 230, the device 200 may be inserted into the cavity and the lid 230 closed to activate the light source(s) 232 to treat the device 200. For example, the cradle 220 may include a sensor (not shown) coupled to the control circuit 228 to detect when the lid 230 is closed (and a device 200 is located within the cavity 224). When the control circuit 228 confirms that the lid 230 is closed, the control circuit 228 may automatically activate the light source(s) 232, e.g., for a predetermined period of time to treat the device 200. Optionally, the cradle 220 may include a locking mechanism (not shown) that automatically locks the lid 230 once closed, e.g., until the predetermined period of time has passed to ensure that the device 200 has been sufficiently cleaned and/or to prevent inadvertent exposure to the light transmitted by light source(s) 232.

In another example, a light treatment device and cradle may include a magnetic switch or other activation circuit that automatically activates the device upon removal from the cradle. For example, the cradle may include a circuit that generates a magnetic field or other energy, and the device may include a sensor therein that detects the presence and/or absence of the field/energy. Thus, when the device is removed from the cradle, the controller of the device may detect the removal and automatically activate the LEDs, e.g., immediately or after a predetermined time delay. Such a time delay may allow sufficient time to insert the device and/or may allow the controller to confirm whether the device has been placed back into the cradle within the predetermined time, e.g., to prevent accidental activation if the device falls out of the cradle and the like.

In still another example, a device having a motion sensor may be used to activate the device. For example, if the controller detects lack of motion from the motion sensor for a predetermined time threshold, the controller may conclude that the device is in the cradle or otherwise not being used. Once motion is detected, the controller may automatically activate the LEDs, e.g., immediately or after a predetermined delay.

According to one example, the device 200 (or any of the other devices herein) may be useful for the treatment of fungal and bacterial vaginitis, candida, chlamydia, and/or other conditions. In case of bacterial vaginitis, there is no need for the use of additional photo sensitizing agents as bacteria are negatively affected by the light-based therapy of the device 200.

The devices herein may help to eliminate or reduce undesirable microorganisms as an adjunct and forms a basis for the replacement of traditional therapies. The device(s) may also be useful for patients who are interested in non-drug therapies. The patients who cannot tolerate oral or topical azole therapy, as well as immune-compromised patients with recurrent yeast or bacterial infections can be treated with the device(s). Optionally, any of the devices may be used as a cleansing device, e.g., for use prior to pregnancy to help avoid bacterial vaginosis issues which may negatively affect a pregnancy.

According to one example, the devices herein may be useful against fungal as well as bacterial infections. The fungal infection comprises the infection caused by yeast and especially by (but not limited to) Candida albicans while the bacterial infection comprises the infection caused principally by Gardnerella.The patient has to determine first whether he is suffering from a fungal infection or a bacterial infection. This can be determined first through a doctor's test.

According to one example, the device may be sold along with a testing strip, e.g., as part of a kit or system for treatment. The testing strip may be used for the determination of the fungal and the bacterial infection suffered by a patient according to the examples herein. The bacterial as well as fungal infections may be treated using the device 100 as an alternative to drugs, douches or chemicals prescribed by a doctor.

According to another example, in case of fungal infection, the device may be used along with a photo-sensitizer. The photo-sensitizer may be beneficial in cases of yeast infection. The photo sensitizer comprises porfimer sodium (Photofrin), 5-aminolevulinic acid or ALA (Levulan), and methyl aminolevulinate [MAOP] (Metvix).

Optionally, any of the devices herein may also be augmented with the use of a probiotic, e.g., to help replenish essential bacteria and fungi for the microbiome.

According to another example, the device 100 (or any of the devices herein) may provide a low power long duration therapy so as to be safer for the mucosal tissue. For example, the device may be inserted overnight and removed in the morning, i.e., after several hours. Alternatively, the device may be inserted for relatively short duration treatments, e.g., thirty to sixty (30-60) minutes, after which the device may be removed. For example, the light source(s) may be single color or multi-color LEDs, and/or may use pulsed or non-pulsed lights.

In one example, the device 100 may be configured for multiple usages, e.g., such that the device 100 may be cleaned and inserted into the vagina multiple times, e.g., over several days or other course of treatment. Alternatively, the device 100 may be a single-use device, i.e., that may be discarded after being used for one treatment.

For multiple usages, the device 100 may include a rechargeable battery and a cord, which may facilitate the removal of the device from the vagina as well as acts as a connection with a suitable power source in order to recharge the device. Alternatively, as with other devices herein, the device 100 may include an inductive charging circuit (not shown) for wireless inductive charging without a cord.

According to one example, the light therapy device comprises one or more LEDs as light source for impending light on the vaginal walls. The device further comprises a battery housed inside the 100% sealed housing or the LED body. The battery may be connected to and act as a power source to the controller as well as the LEDs. The microchip controls a duration of the light therapy. A printed circuit or a suitable electronic circuitry or hub may be provided in the device for interconnecting the switch, the LEDs, the microchip and the battery. The device further comprises switch activates a device to start the light therapy. The device also comprises a tether for retrieval of the device during a light therapy. The tether is suitably replaced by a USB cord or a charging cord for making device suitable for multiple usage.

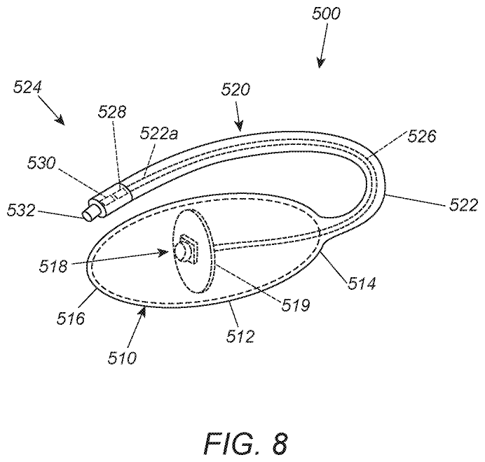

Turning to FIG. 8, another example of a light therapy device 500 is shown that is constructed generally similar to the previous examples, e.g., including an oblong-shaped body 510 containing internal components of the device 500 within a sealed environment and a flexible tether 520 extending from one end of the body 510. Similar to other examples herein, the body 510 may include a central region 512 defining a maximum diameter that tapers to proximal and distal ends 514, 516, e.g., having rounded proximal and distal ends 514, 516 such that the body 510 defines an ellipsoid shape.

In addition, the device 500 includes one or more light sources, e.g., one or more LEDs 518 mounted on a circuit board or other substrate 519 within the body 510 configured to deliver light to tissue surrounding the body. For example, the body 510 may include a plurality of lenses, windows or other transparent features (not shown) spaced apart from one another in the wall of the body 510 to deliver light from the LED(s) 518 through the wall to the surrounding tissue. Alternatively, a plurality of LEDs or other light sources may be mounted directly in the wall of the body 510 (not shown).

Unlike previous examples, the tether 520 includes an elongate member 522 carrying an outer housing 524, e.g., on a free end 522a of the elongate member 522. The outer housing 524 may include one or more components of the device 500, e.g., a battery 528 for delivering power to the LED(s) 518. A controller 530 may also be provided in the outer housing 524 coupled to the battery and to the LED(s) 518, e.g., via one or more wires or other leads 526 extending from the body 302 through the elongate body 322 to the outer housing 324. Alternatively, the controller may be located within the body 510, e.g., mounted on a circuit board including the LED(s) 518. Alternatively, the outer housing 524 may be separate from the elongate member 522 and the free end 522a and housing 524 may include a connector for removably coupling the housing 524 to the elongate member 522, e.g., before or after inserting the body 510 into the vagina, e.g., an electrical and/or mechanical connector for coupling the components within the housing524 to the lead 526.

Optionally, the device 500 may also include a wireless communications interface 532, e.g., a transmitter/receiver communicating via radiofrequency (RF) signals, e.g., using Bluetooth or other wireless protocols. For example, the interface 532 may be coupled to the controller 530, e.g., to allow activation and/or deactivation of the device 500 remotely, e.g., using an application on a cellphone or other electronic device, similar to other examples herein. Alternatively, the outer housing 524 may include a switch (not shown) for activating and/or deactivating the LED(s) 518 during use.

In another option, the elongate member 522 of the tether 520 may be malleable, e.g., sufficiently flexible that the tether 520 may be manipulated by the user but substantially maintain a shape applied to the tether 520, e.g., a curved shape as shown in FIG. 8, which may facilitate positioning the outer housing 524 at a desired location when the body 510 is inserted into the user's vagina during a treatment to minimize discomfort. Alternatively, the tether 520 may be biased to a predetermined shape, e.g., the curved shape shown, but may be sufficiently flexible to facilitate manipulation of the tether 520, e.g., to facilitate insertion and removal of the body 510. In yet another alternative, the tether 520 may be substantially rigid, e.g., set to curved or other desired shape.

The device 500 may be used similar to other examples herein, e.g., by inserting the body 510 entirely into the user's vagina, e.g., until the distal end 516 is positioned adjacent the cervix, and the tether 520 extends from the vagina. The user may then activate the LED(s) 518, e.g. remotely or by manipulating a switch on the outer housing 524. If malleable, the elongate member 522 of the tether 520 may be manipulated to move the outer housing 524 to a desired location, e.g., against the user's body adjacent the entrance to the vagina, to minimize discomfort during treatment.

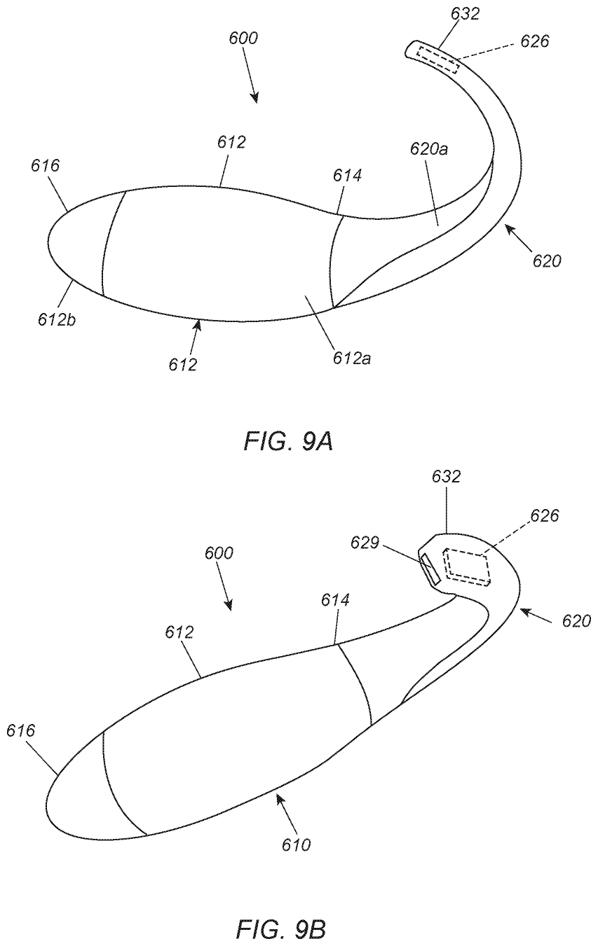

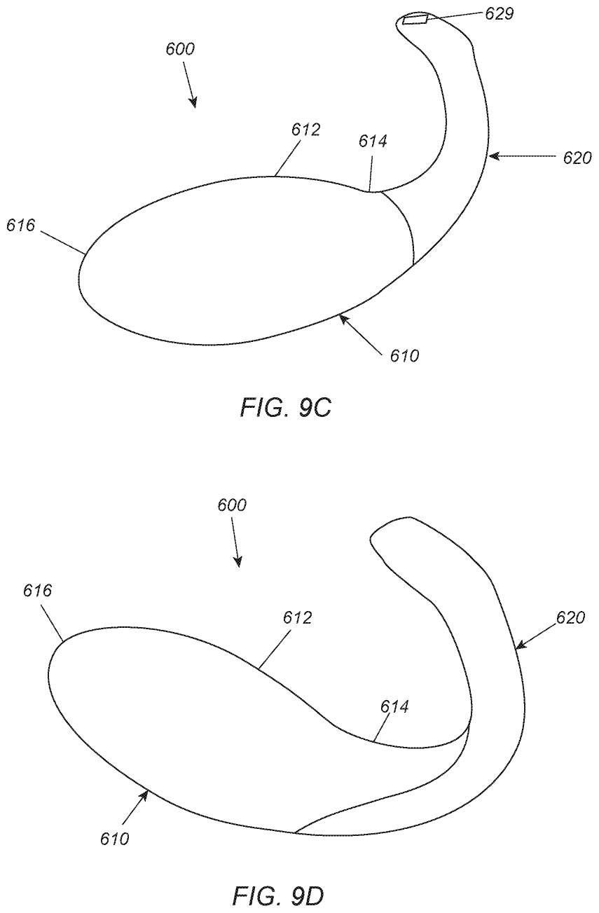

Turning to FIGS. 9A-9D, another example of a light therapy device 600 is shown that is constructed generally similar to the previous examples, e.g., including an elongate body 610 sized for insertion into a user's vagina for delivering light therapy. In the example shown, the body 610 includes a central region 612 defining a maximum diameter that tapers towards proximal and distal ends 614, 616 of the body 610, e.g., a having rounded distal end 616 such that the body 610 generally defines an ellipsoid shape. Such an ellipsoid and/or other rounded shape may facilitate easy entry into the vagina and/or enhance comfort for the user, similar to other examples herein. In addition, the device 600 also includes a tail or appendage 620 extending from the proximal end 614 of the body 610, e.g., terminating in a proximal tip 632 of the device 600, which may remain outside the user's vagina during use.

The device 600 includes one or more electrical components, e.g., mounted on a circuit board or flexible PCB substrate 626 within the appendage 620, e.g., adjacent the proximal tip 632, as shown in phantom in FIGS. 9A and 9B. Similar to previous examples, the device 600 includes one or more light sources 618 configured to deliver light to tissue surrounding the body. In one example, the light source(s) 618 may include one or more LEDs mounted on the substrate 626 that deliver light into the body 610, e.g., via one or more optical fibers (not shown) into the body 610. Alternatively, the light source(s) 618 may be located directly within the body 610, e.g., on one or more additional substrates mounted within the body 610, e.g., similar to the device 500 shown in FIG. 8 and/or other examples herein.

For example, the body 610 may include a plurality of lenses, windows or other transparent features (not shown) spaced apart from one another in the wall of the body 610 to deliver light from the light source(s) 618 through the wall to the surrounding tissue. Alternatively, a plurality of LEDs or other light sources may be mounted directly in the wall of the body 610 (not shown). In a further alternative, one or more regions of the body 610 may be translucent or transparent to light transmitted by the light source(s) 618 outwardly from the interior of the body 610.

The appendage 620 may be integrally formed with the body 610 to provide a unitary housing for the device 600, e.g., by one or more of molding, casting, machining, or otherwise formed together from plastic, metal, and/or composite materials. The appendage 620 may have sufficient length and a desired shape that extends proximally from the body 610 to facilitate manipulation of the device 600, e.g., during insertion and/or removal. For example, the appendage 620 may have a length longer than the body 610. Optionally, the material of the appendage 620 may be malleable, e.g., sufficiently flexible that the appendage 620 may be manipulated by the user but substantially maintain a shape applied to the appendage 620, e.g., a curved shape as shown in FIGS. 9A-9D, which may facilitate positioning the appendage 620 at a desired location when the body 610 is inserted into the user's vagina during a treatment. Alternatively, the appendage 620 may be biased to a predetermined shape, e.g., the curved shape shown, but may be sufficiently flexible to facilitate manipulation of the appendage 620, e.g., to facilitate insertion and removal of the device 600. In this alternative, once released, the appendage 620 will automatically return towards the predetermined, e.g., curved, shape. In yet another alternative, the appendage 620 may be substantially rigid, e.g., set to curved or other desired shape.

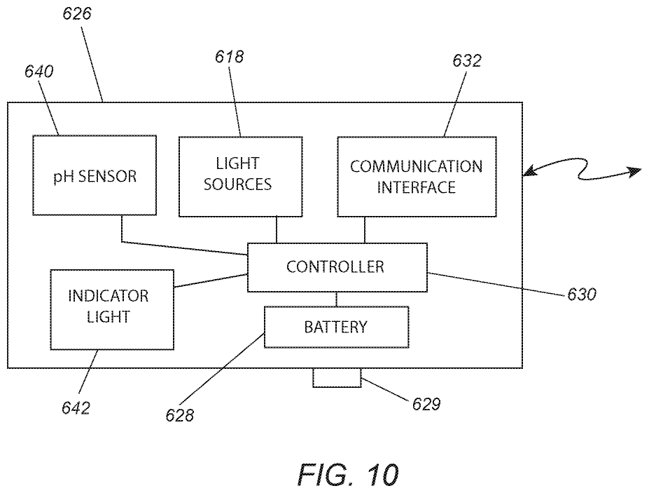

Turning to FIG. 10, exemplary components are shown that may be carried on the substrate 626 or otherwise within the appendage 620, e.g., to position the components outside the user's body when the body 610 of the device 600 is inserted into the user's vagina. For example, a battery 628 may be provided for delivering power to the light sources, and a controller 630 may be coupled to the battery 628 and the light source(s) 618 to control operation of the light source(s) 618. Optionally, a wireless communications interface 632 may also be provided, e.g., a transmitter/receiver communicating via radiofrequency (RF) signals, e.g., using Bluetooth or other wireless protocols, similar to other examples herein. For example, the interface 632 may be coupled to the controller 630, e.g., to allow activation and/or deactivation of the device 600 remotely, e.g., using an application on a cellphone or other electronic device, similar to other examples herein. Alternatively, the appendage 620 may include a switch (not shown) and/or use motion controls for activating and/or deactivating the device 600 during use, similar to other examples herein.

The appendage 620 may include a connector 629, e.g., on or adjacent the proximal tip 632, which may be coupled to the battery 628 and/or controller 630, e.g., such that a cable (not shown) may be removably connected to the device 60 to recharge the battery 628 and/or program the controller 630. Alternatively, the device 600 may include an inductive charging circuit within the appendage 620, similar to other examples herein, and the connector 629 may be omitted.

The device 600 may be used similar to other examples herein, e.g., by inserting the body 610 into a user's vagina, e.g., until the distal end is positioned adjacent the cervix, and the appendage 620 extends from the vagina (not shown). The user may then activate the light source(s) 618, e.g., remotely or by manipulating a switch on the appendage 620. If malleable, the appendage 620 may be manipulated to move the appendage 620 to a desired location, e.g., against the user's body adjacent the entrance to the vagina, to minimize discomfort during treatment.

Optionally, the device 600 may include different regions that may include separate light sources and/or that may be activated independently, e.g., separately or simultaneously, e.g., for treating one or more areas of the user's body including the introital region and/or external genitalia. For example, as shown in FIG. 9A, the body 610 may include an annular region 612a, e.g., extending distally from the appendage 620 to a distal region 612b of the body 610, that may be include one or more light sources that may be activated to deliver light outwardly along the annular region 612a for treating the vaginal cavity. Optionally, the distal region 612b may also include one or more light sources that may be activated independently to deliver light distally from the body 610, e.g., towards the user's cervix.

In addition or alternatively, the appendage 620 may include one or more regions that include one or more additional light sources that may be activated for treating the vaginal canal and/or external regions, e.g., the vulva, labia majora, and/or labia minora, e.g., to treat vulvo vaginitis. In the example shown, the appendage 620 includes a proximal region 620a extending at least partially along an upper, concave side of the appendage 620, which may allow treatment of external anatomy.

In one example, a single set of LEDs may be provided on the substrate 626 whose light may be split and delivered to the respective regions via one or more optical fibers, lenses, and the like, e.g., to deliver light sequentially, simultaneously, and/or otherwise to the different regions. Alternatively, separate light sources may be provided within each of the regions, which may be separated from one another by partitions to prevent light from leaking from one region to another. The separate light sources may again be activated sequentially, simultaneously, and the like. For example, with the body 610 fully inserted into the user's vaginal cavity and the appendage 620 extending proximally out of the vagina, the user may selectively activate the device 600 to deliver light within vaginal cavity via the body 610 and/or along the canal and/or external genitalia via the region(s) on the appendage 620.

Optionally, the device 600 (or any of the other devices herein) may include one or more sensors 640, e.g., carried on the body 610 and/or appendage 620. For example, a pH sensor 640 may be provided on the body 610 that is coupled to the controller 630, which may process signals from the pH sensor 640 to identify one or more conditions. For example, if the controller 630 detects that the pH within the user's vagina is greater than 4.5 (or other threshold value), the controller 630 may conclude that the user has bacterial vaginosis or Thrichomonas organisms present. Optionally, the device 610 may include an output device 642, e.g., an indicator light, display, and the like coupled to the processor 630, which may provide an output to inform the user, e.g., by changing color or displaying a text message. For example, the light 642 may be mounted on the appendage 620 of the device 610 such that the user may readily see an indication when the light 640 is activated by the processor 630 during use of the device 610. Alternatively, a light may be provided on a tether, cable or other component extending from the body of a therapy device, such as on the tether 105 or cable 106 of the devices shown in FIGS. 1A-11F, 3, and 8.

In addition or alternatively, one or more temperature sensors (not shown) may be provided on the device 610 (or any of the other devices), e.g., coupled to the controller 630, to detect basal temperature within the user's vagina. For example, a temperature sensor carried on an outside surface of the device 610 may be coupled to the processor 630 to provide signals that maybe analyzed by the processor 630 to monitor one or more conditions of the user. In one example, the processor 630 may monitor the user's temperature to track the user's menstrual cycle, e.g., by tracking the user's basal body temperature. In addition or alternatively, other sensors (also not shown) may be provided on the device 610 to assist such analysis and/or diagnosis.

In addition or alternatively, if the device 610 includes a communications interface 632, data (or the analysis by the controller 730) from the pH sensor, temperature sensor, and the like may be communicated to an external device, e.g., a mobile phone, tablet, or other electronic device including an application that may inform the user. Optionally, the application on the electronic device may provide additional interfaces and/or functionality to the user and/or may communicate information related to the user, e.g., an indication of bacterial vaginosis, to the user's health care provider, e.g., to record the information in the patient's health file and/or facilitate the provider treating the user.

Optionally, such an application may provide additional guidance to the user, e.g., related to frequency and/or duration of user of the device 610, e.g., based on the pH detected and/or diagnosis of bacterial vaginosis or other condition.

In addition, if the device 610 includes a temperature sensor, the processor 630 may provide such information to the electronic device, e.g., to a software application that may provide output to the user regarding their basal body temperature, e.g., to facilitate monitoring ovulation for pregnancy guidance and the like.

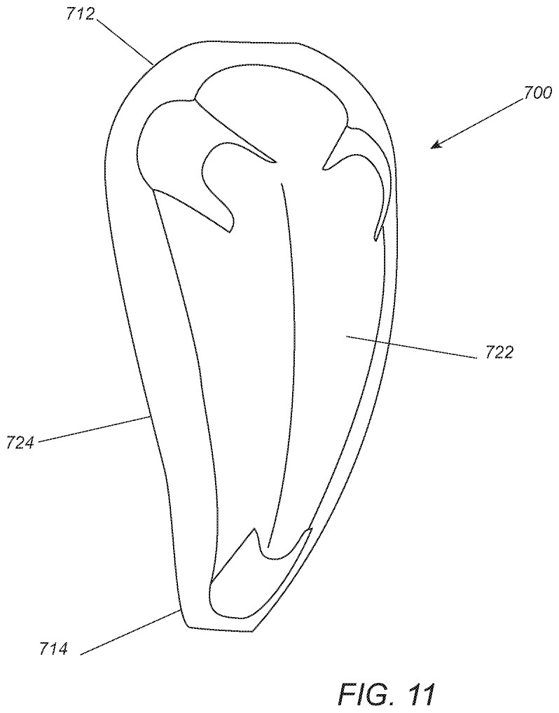

FIG. 11 shows another example of a light therapy device that may be configured for placement externally adjacent the vagina, e.g., to provide therapy to external surfaces of the vagina, e.g., to treat vulvo vaginitis. Unlike the previous examples, the device 700 may include one or more light sources, e.g., LED(s) and/or windows, along its concave surface, which may be positioned against the user's external anatomy and activated for light treatment. In addition, the device 700 may include one or more other components, similar to other examples herein, e.g., a controller, a battery, a wireless communications interface, an inductive charging circuit, a connector, and the like (not shown).

The device 700 includes a body have a fixed shape, e.g., including an upper region 712 and a lower region 714 that tapers from the upper region 712. The lower region 714 may have a tapered and/or pronounced shape and/or tip, which may be configured to separate the labia to facilitate exposure of the vulva for light therapy. Thus, while holding the upper region 712, the lower region 714 of the device 700 may be inserted between the user's labia to expose the vulva, whereupon one or more light sources may be activated to deliver light therapy to the vulva and/or other exposed regions. As shown, the body may include a slightly convex outer surface 722 and a slightly concave inner surface 724, e.g., shaped to position the device against the user's pubic region. Optionally, the light source(s) may emit light only from the inner surface 722, e.g., along the lower region 714 to deliver light therapy to the exposed anatomy.

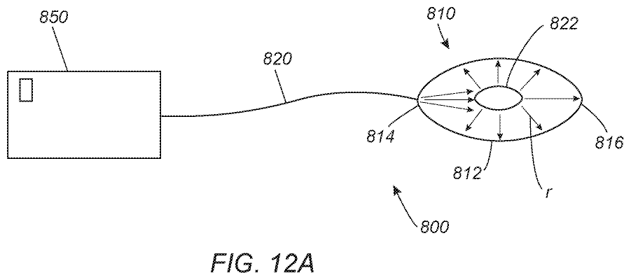

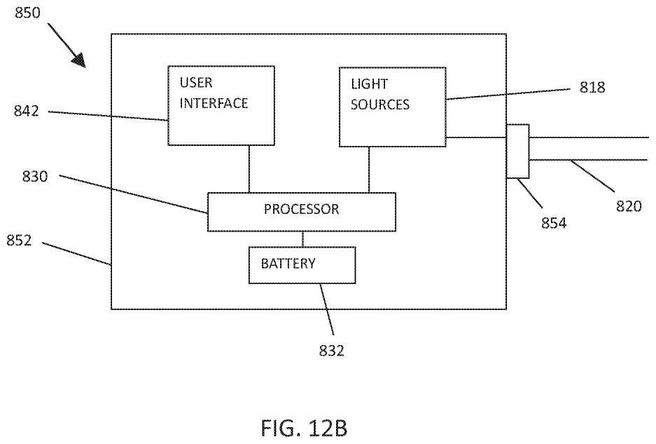

Turning to FIGS. 12A-12B, another example of a light-based therapy system is shown that includes a light therapy device 800, which may be constructed generally similar to the previous examples, and an external controller 850. For example, as shown in FIG. 12A, the device 800 may include an elongate body 810 sized for insertion into a user's vagina for delivering light therapy. In the example shown, the body 810 includes a central region 812 defining a maximum diameter that tapers towards proximal and distal ends 814, 816 of the body 810, e.g., having rounded proximal and distal ends 814, 816 such that the body 810 generally defines an ellipsoid shape. Such an ellipsoid and/or other rounded shape may facilitate easy entry into the vagina and/or enhance comfort for the user, similar to other examples herein. Optionally, the device may also include a tether, cable, tail and/or appendage (not shown) extending from the proximal end 814 of the body 810, e.g., which may remain outside the user's vagina during use, similar to other examples herein.

In the system shown, the device 800 does not include an internal light source and, instead, as shown in FIG. 12B, one or more LEDs or other light sources 818 are provided within the controller 850, which may include a processor and/or other electronics 830 to operate the device 800. Such external light source(s) 818 may reduce heat generated within the body 810, which may increase comfort to the subject using the device 800. For example, a fiberoptic cable 820 may extend from the controller 850 to the proximal end 814 of the body 810, which may include one or more optical fibers for delivering light from the light source(s) 818 into the body 810 for delivery outwardly from the device 800. The light source(s) 818 may include one or more LEDs, OLEDs, and the like.

In one example, the cable 820 may include a plurality of optical fibers, which may be optically coupled to respective fibers within the body 810 that include ends secured to the body 810, e.g., to the inner surface of the body 810 for delivering light through respective lenses, filters, and the like mounted on the wall of the body 810. For example, light from the source(s) 818 may be delivered through each individual fiber through a respective lens to deliver the light into a subject's vagina.

Alternatively, as shown in FIG. 12A, the one or more optical fibers may be optically coupled to a ray shaper 822 mounted within the body 810, which may be configured to split the incoming light into a plurality of beams and/or otherwise disperse the incident light, which may be transmitted radially outwardly from the ray shaper 822. In this alternative, the body 810 may be substantially transparent (at least within the wavelengths of light transmitted by the light sources 818) such that the light passes through the body 810 and outwardly to the surrounding tissue, e.g., as represented by light rays “r.” In this alternative, the cable 820 may include a single or multiple optical fibers, as desired, such that the light may be delivered to and transmitted by the ray shaper 822.

The cable 820 may be permanently connected to the body 810 or, alternatively, the body 810 may include a connector (not shown) to which one end of the cable 820 may be coupled to deliver the light from the source(s) 818. Similarly, the cable 820 may be permanently connected to the controller 850 or, alternatively, the controller and cable may include mating connectors to allow the cable 820 to be removably coupled to the controller 850, e.g., connector 854 shown in FIG. 12B.

Optionally, the controller 850 may include one or more additional components, e.g., a battery 832 or other power source, which may operate the electrical components of the controller 850. In addition or alternatively, the controller 850 may include a cable for connecting to an external power source (not shown), e.g., to charge the battery 832 and/or power the controller 850 directly. In addition or alternatively, the controller 850 may include an inductive charging circuit (not shown), e.g., similar to other devices described elsewhere herein.

Further, optionally, the controller 850 may include a user interface 842, e.g., an output device, e.g., a display, keypad, and other types of interactive interfaces. For example, the user interface 842 may include one or more input devices, such as a microphone, keyboard, touchscreen, and the like, and one or more output devices, such as a display, speaker, light indicators, and the like.

Further optionally, the controller 850 may include memory, e.g., coupled to the processor 840 to store data related to operation of the device 800 and/or date related to one or more sensors (not shown) if provided on the device 800, e.g., similar to other devices herein. The controller 850 may also include a wireless communications interface, e.g., for receiving instructions and/or for communicating information to a remote electronic device, similar to other devices herein.

Optionally, the device 800 may include one or more sensors (not shown), e.g., a pH sensor, temperature sensor, and the like, similar to other devices herein. Optionally, the device 800 may include one more additional components providing additional functionality. For example, the device 800 may include a vibration generator, which may be activated by the user via the controller 850 to generate vibrations, e.g., to facilitate dilating tissue and/or exposing tissue to light emitted by the device 800.

In one example, the components of the controller 850 may be contained within a housing 852 to provide a lightweight, portable controller 850 that may be carried and/or worn by a user. For example, the housing 852 may include one or clips, straps, and the like (not shown) such that the controller 850 may be secured to the user and/or their clothing. For example, the user may insert the device 800 into their vagina and secure the controller 850 to their undergarments or other article of clothing, secure a strap around their leg or waist, thereby allowing the device 800 to provide light therapy discretely. The user may program or manually activate the device 800 as desired, e.g., using the user interface 842 of the controller 850. Optionally, information may be provided via the controller 850 and/or communicated to a remote electronic device, which may present information to the user related to use of the device 800, similar to other deices herein.

According to the examples herein, light-based therapy devices are provided that may be useful to kill or render inert the targeted species which keeps the species from replicating. The device(s) may also be used as an adjunct therapy with existing known treatments possibly allowing for a reduction in drug or chemical based therapies. If the device is used with the conventional therapies, then the device is likely to reduce the treatment times.

Optionally, the effectiveness of any of the devices herein may be enhanced through the use of a photo-sensitizer. For example, a photo-sensitizer may be applied to the outer surface of the body 102 or into pockets or features (not shown) configured for receiving the photo-sensitizer. Optionally, the outer surface of the device(s) may include one or more features, e.g., pockets, depressions, and the like spaced apart from one another on the outer surface of the device, into which photo-sensitizer and/or other medications or agents may be applied before use. Such pockets, depressions, or other features may facilitate delivering one or more agents distributed over the surface of the device while minimizing the agents being wiped off during insertion. Alternatively, the photo-sensitizer or other agents may be introduced separately into the vagina, e.g., using known applicators (not shown). The device(s) may be used in conjunction with standard systemic drug or topical cream-based therapies to lessen the duration of the event.

For example, a photosensitizer that includes curcumin may be used with any of the light-based therapy devices described herein, e.g., applied directly to the outer surface of the device and/or delivered independently but in conjunction with using the device. In one example, curcumin may be mixed with a carrier agent to create a water soluble material for use as photosensitizer. The material may be applied to the outer surface of the device, e.g., into recesses in the outer surface, as described previously, before introduction of the device into the user's vagina.

Examples of carrier agents that may be used include dimethylsulfoxide (DMSO), carboxy methyl cellulose (CMC), and the like. In one example, a photosensitizer may be provided that includes a CMC carrier with sufficient curcumin mixed or carried in suspension with the carrier to provide a concentration of not more than about two millimolar (2.0 mM), or between about half and two millimolar (0.5-2.0 mM), or between about one and ten micromolar (1.0-10.0 um). Optionally, the carrier may also provide a lubricant, which may facilitate introduction and/or other reduce discomfort during use of the device.

Curcumin may enhance light-based therapies of such pathogens as Candida albicans, Gardnerella vaginalis, and the like, e.g., improving one or more parameters of treatment compared to devices within curcumin, such as lowering power requirements, duration of treatment sessions, and/or overall dosage of light delivered to the user. For example, where previous light-based therapy devices were found to be more effective at wavelengths of about 405 nanometers, using a curcumin-based photosensitizer, light wavelengths may be shifted further away from the near-ultraviolet wavelengths. For example, wavelengths between about 425 and 475 nanometers, about 430-450 nanometers, and/or a narrow band centered around between 445 and 450 nanometers may be used to effectively treat target pathogens without significantly affecting other desirable bacteria, such as Lactobacillus, that may be present within the vagina. In particular, light at wavelengths between about 445-450 nanometers may maximize activation of curcumin to enhance treatment of vaginal infections.

Further, it has been found that shifting the light wavelengths of the therapy devices may allow overall power to be substantially reduced, e.g., up to seventy five percent less, compared to previous light-based therapy devices. For example, where it was previously found that power levels of fifty to sixty milliwatts per square centimeter of surface area of the therapy device (50-60mW/cm2) were needed for sufficient light treatment, curcumin-based light therapies may be effective with the device delivering under fifty milliwatts per square centimeter (50 mW/cm2), e.g., not more than about twenty five milliwatts per square centimeter of surface area of the therapy device (25 mW/cm2), or not more than about twenty milliwatts per square centimeter (20 mW/cm2), or not more than about ten milliwatts per square centimeter (10 mW/cm2). For some pathogens, power levels of between about five and six milliwatts per square centimeter (5-6 mW/cm2) may be sufficient to treat pathogens, substantially reducing power requirements of the therapy device, which may reduce heat that may otherwise create discomfort for the user, reduce treatment times, and/or overall dosage.

Optionally, the devices may be configured to deliver light therapy in pulses rather than substantially continuously once activated, which may enhance activation of the curcumin-based photosensitizer, while also reducing power consumption and/or heat generation during use. For example pulses having a duty cycle of no more than fifty percent (50%) may deliver sufficient light in combination with a curcumin-based photosensitizer to effectively treat various pathogens.

It is to be understood that the phraseology or terminology employed herein is for the purpose of description and not of limitation. Therefore, while the examples herein have been described in terms of exemplary embodiments, those skilled in the art will recognize that the examples herein can be practiced with modification within the scope of the claims.

1. A device for vaginal light therapy of a user, comprising:
a housing comprising a body sized for introduction into a vagina;
one or more light sources configured to emit light outwardly from the body; and
a source of photosensitizer comprising curcumin.

2. The device of claim 1, wherein the housing comprises an outer surface configured for receiving the photosensitizer.

3. The device of claim 2, wherein the outer surface comprises a plurality of pockets or depressions configured to receive the photosensitizer.

4. The device of claim 1, wherein the photosensitizer has a concentration of curcumin of not more than about two millimolar (2.0 mM).

5. The device of claim 4, wherein the concentration of curcumin is between about half and two millimolar (0.5-2.0 mM).

6. The device of claim 1, wherein the one or more light sources are configured to deliver light at wavelengths between about 425 and 475 nanometers.

7. The device of claim 6, wherein the one or more light sources are configured to deliver light at wavelengths between about 440-450 nanometers.

8. The device of claim 6, wherein the one or more light sources are configured to deliver light at a narrow band centered around 445-450 nanometers.

9. The device of claim 1, wherein the one or more light sources are configured to generate power under fifty milliwatts per square centimeter of surface area of the body (50 mW/cm2).

10. The device of claim 9, wherein the one or more light sources are configured to generate power of not more than about twenty five milliwatts per square centimeter (25 mW/cm2).

11. The device of claim 9, wherein the one or more light sources are configured to generate power of not more than about ten milliwatts per square centimeter (10 mW/cm2).

12. The device of claim 9, wherein the one or more light sources are configured to generate power of between about five and six milliwatts per square centimeter (5-6 mW/cm2).

13. The device of claim 1, wherein the photosensitizer comprises a carboxy methyl cellulose (CMC) carrier.

14. The device of claim 14, wherein the curcumin is mixed or in suspension with the CMC carrier.

15. A device for vaginal light therapy of a user, comprising:
a housing comprising a body sized for introduction into a vagina;
one or more light sources configured to emit light outwardly from the body; and
a source of photosensitizer comprising a carboxy methyl cellulose (CMC) carrier and curcumin mixed or in suspension with the CMC carrier, the concentration of curcumin being not more than about two millimolar (2.0 mM).

16. The device of claim 15, wherein the concentration of curcumin is between about half and two millimolar (0.5-2.0 mM).

17-24. (canceled)

25. A method for vaginal light therapy of a user, comprising:
providing a therapy device comprising body comprising one or more light sources;
providing a photosensitizer comprising curcumin on or in conjunction with the body;
inserting the therapy device into a vagina; and
activating the one or more light sources to emit light outwardly from the body.

26. The method of claim 25, wherein providing the sensitizer comprises applying the photosensitizer to an outer surface of the body before inserting the therapy device into the vagina.

27. The method of claim 25, wherein the photosensitizer has a concentration of curcumin of not more than about two millimolar (2.0 mM).

28. The method of claim 27, wherein the concentration of curcumin is between about half and two millimolar (0.5-2.0 mM).

29-33. (canceled)
